(19) United States
(12) Patent Application Publication
(10) Pub. No.: US 2008/0152537 A1
(43) Pub. Date: Jun. 26, 2008

(54) Sterilization System

(76) Inventors: Hans-Peter Wild, Eppelheim (DE); Wolfgang Tilz, Schwetzingen (DE)

Correspondence Address: STROOCK & STROOCK & LAVAN LLP, 180 MAIDEN LANE, NEW YORK, NY, 10038, US

(21) Appl. No.: 11/935,984

(22) Filed: Nov. 6, 2007

(30) Foreign Application Priority Data
Nov. 7, 2006 (EP) 06023159.4

(51) Int. Cl. A61L 2/20

(52) U.S. Cl. 422/28; 422/83

(57) ABSTRACT

A sterilization system is provided for sterilizing a connection area of a bag-in-tank system for the transport of food products, wherein the bag-in-tank system includes a tank, a selectively removable liner having an outlet connector, and a selectively removable valve connectable to the outlet connector. The sterilization system preferably includes a first sterilization station for sterilizing the valve, wherein the valve is disconnected from the tank, and a second sterilization station for sterilizing the valve and the outlet connector in an assembled state.

### BACKGROUND OF THE INVENTION

This invention relates, in general, to a system and a method for sterilizing a connecting area of a bag filling system, for example, a bag-in-tank system for transporting food products. More specifically, the system can be suitable for bag-in-tank systems having a removable liner, also referred to as an inliner, inside a tank, an outlet connection and a removable valve assembly.

Built-in-tank systems are often used to transport food products, such as fluid or flowing food products. Examples of transported food products include drinks and fruit preparations. One benefit that bag-in-tank systems provide is the ease of cleaning the system between uses. Rather than the laborious cleaning required in conventional transport tanks, the liner of a bag-in-tank system can be removed and replaced with a new liner. Whereas the system is designed such that the food product contacts the inside of the liner and not the inside of the tank, the tank is often disinfected and sterilized nonetheless as a precaution. For example, when products such as fluid or flowing food products are being transported, a higher hygiene requirement is often required to guarantee a package that is substantially free of germs. Therefore, the connection area via which the liner is filled or emptied is often cleaned and sanitized.

In light of the shortcomings described above, it is desirable to provide a system for sterilizing the connection area of a bag-in-tank system, preferably which meets the higher hygiene requirements associated with systems for transporting food products.

### SUMMARY

Generally speaking, the present invention is directed toward a sterilization system suitable for use with a bag-in-tank system having a tank and a removable liner for receiving a product being transported. The sterilization system preferably includes a plurality of sterilization stations. A first sterilization system can have a docking device and a holding device for receiving a valve therebetween. The docking device can be movable toward the valve and can form a sealed unit with the valve and the holding device, after which sterilization can be performed. A plurality of testing mechanisms can be provided in an analysis chamber, such as a pressure measuring device, a temperature sensor and a discharge for receiving condensation. Such testing mechanisms preferably assure that proper sterilization is being performed.

Compressed air can be released into the docking device-valve-holding device assembly, after which the assembly can be sterilized. Sterilization can be performed by releasing water vapor or other suitable sterilizing agent into the assembly.

In accordance with an embodiment, a common docking device can be pivoted between the first and second sterilization stations. The second sterilization station can have a docking device for connecting the valve to the bag-in-tank system and sterilizing the connection area formed between the valve and the corresponding outlet connector of the liner of the bag-in-tank system. Hydrogen peroxide or other suitable sterilizing agent can be introduced into the connection area at the second sterilization station.

A third sterilization station can be provided for sterilizing the connection area again prior to filling the liner. In accordance with an embodiment, the third sterilization station can be at the filling station, wherein a common docking device is used to provide the sterilizing agent into the connection area as well as to supply the product to the liner.

An object of the invention is to provide an improved sterilization system as described herein.

Other objects and features of the present invention will become apparent from the following detailed description, considered in conjunction with the accompanying drawing figure. It is to be understood, however, that the drawings are designed solely for the purpose of illustration and not as a definition of the limits of the invention, for which reference should be made to the appended claims.

### BRIEF DESCRIPTION OF THE FIGURES

For a fuller understanding of the invention, reference is had to the following description, taken in connection with the accompanying drawings, in which:

FIG. 1 is a cross-sectional view of a portion of a system in accordance with an embodiment of the invention;

FIG. 2 is a schematic view of a sterilization station of a sterilization system in accordance with an embodiment of the invention;

FIG. 3 is a schematic view of a sterilization station of a sterilization system in accordance with an embodiment of the invention;

FIG. 4 is a schematic view of a sterilization system in accordance with an embodiment of the invention;

FIG. 5 is a flowchart of a method for sterilizing in accordance with an embodiment of the invention; and

FIG. 6 is a flowchart of a method for sterilizing in accordance with an embodiment of the invention.

### DETAILED DESCRIPTION

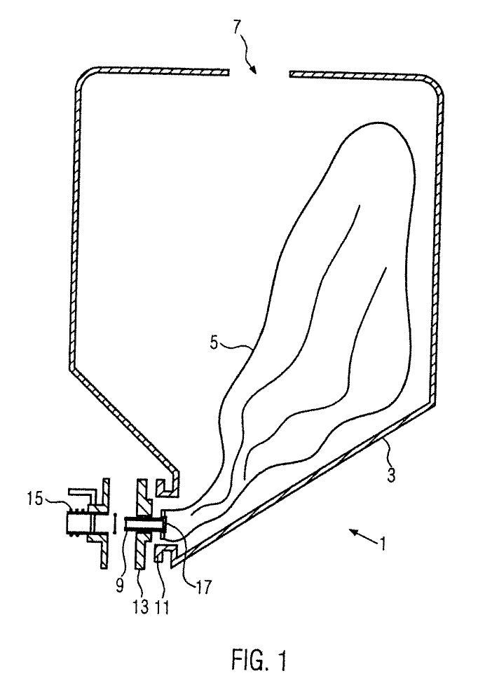

Reference is made to FIGS. 1-4, wherein an embodiment of a sterilization system is illustrated. FIG. 1 shows an embodiment of a bag-in-tank system generally indicated at 1 for transporting food products, by way of non-limiting example, juice and fruit preparations. Bag-in-tank system 1 includes a tank 3 constructed and arranged to receive a liner 5 therein, wherein liner 5 can be filled with the food product being transported. After use, liner 5 can be removed and replaced with a new liner 5, for example, through an opening 7 in tank 3. Alternatively, liner 5 can be removed and inserted through outlet opening 11, in accordance with application specific design choice. Liner 5 preferably includes an outlet connector 9 constructed and arranged to protrude through an outlet opening 11 of tank 3.

A washer 13 and valve 15 can be provided to create a seal with outlet opening 11 of tank 3, wherein washer 13 can be positioned between valve 15 and tank 3. Washer 13 preferably includes an opening (not shown) through which outlet connector 9 can be received, preferably centering outlet connector 9 within outlet opening 11 and aligning outlet connector 9 with valve 15 such that the outer end of outlet connector 9 can be received by valve 15.

In accordance with an embodiment, outlet connector 9 includes a bursting element 17, such as a bursting diaphragm or disk. Bursting element 17 preferably covers, more preferably seals, the opening of outlet connector 9, and can be located inside liner 5. As the product is being introduced into liner 5, bursting element 17 can burst open to permit the product to pass through outlet connector 9 and into liner 5. Preferably, bursting element 17 does not shred, thus preventing pieces of bursting element 17 from being mixed with the product in liner 5.

As used herein, “connection area” refers to the interior area spanning the length of valve 15 and the length of outlet connector 9 up to bursting element 17. In accordance with an embodiment of valve 15, valve 15 can be connected to outlet connector 9 either alone or with tank 3. For example, valve 15 can be connected to tank 3 via gripper clamps or other securing elements. An embodiment of the sterilization system can clean, preferably sterilize, the connection area as well as the air therein.

FIG. 2 illustrates an embodiment of a first sterilization station 21 of the sterilization system, at which valve 15 can be sterilized. As shown, valve 15 is preferably detached from tank 3 while being sterilized at first sterilization station 21. First sterilization station 21 can include a holding device 23 and a docking device 25. Holding device 23 is preferably constructed and arranged to retain valve 15 as valve 15 is being sterilized. Providing holding device 23 preferably facilitates proper handling of valve 15 and reduces the potential for handling and operating errors.

Holding device 23 can include a holder 27 for receiving valve 15 and a holder conduit 29 aligned with valve 15 when valve 15 is received by holder 27. Holder conduit 29 can be connected to an analysis chamber 31, which is preferably connected to a pressure measuring device 33 and a temperature sensor 35. A discharge valve 37 is preferably connected to analysis chamber 31, wherein discharge valve 37 can block or open a discharge 39.

Docking device 25 can include a connecting assembly 41 which can form a sealed unit with valve 15 and holding device 23. Connecting assembly 41 preferably includes one or more grippers (not shown) to apply a force on connecting assembly 41, valve 15 and holding device 23 toward each other to create the sealed unit. Docking device 25 preferably also includes a docking conduit 43 aligned with valve 15 and holder conduit 29 when assembled. By providing a sealed unit, which is preferably leak-proof, the sterilization process can preferably be monitored.

For example, compressed air can be introduced into the sealed unit to determine whether or not the tightness of docking device 25, valve 15 and holding device 23 is sufficient. The leakage test preferably ensures that sterilization is performed with minimal errors to assure compliance with the hygiene requirements. Additionally, if a hot vapor is used for sterilization, the vapor preferably condenses on the cold surfaces of valve 15, and the temperature of the condensate can provide information regarding the temperature of the walls. It can thus be determined whether or not the sterilization process has been effectively performed. A continuous temperature check can also be used to verify that the sterilization was carried out for a sufficient length of time at the required temperature.

Referring to FIG. 2, docking device 25 can include a plurality of hinged joints 45a, 45b, which can facilitate docking device 25, more particularly connecting assembly 41, being moved toward valve 15 and holding device 23. It is to be understood that any other suitable means for displacing connecting assembly 41 toward valve 15 and holding device 23 can be provided without deviating from the scope of the invention. Docking device 25 preferably has an end 47 facing away from valve 15, end 47 having a first connection 49 and a second connection 51. A vapor feeding unit 53 for supplying vapor, by way of non-limiting example, water vapor, is preferably connected to docking device 25 at first connection 49 via a first valve 55. A compressed air feeding unit 57 is preferably connected to docking device 25 at first connection 49 via a second valve 59. A sterilizing agent feeding unit, for example, a hydrogen peroxide feeding unit 63 is preferably connected to docking device 25 at second connection point 51 via a third valve 61.

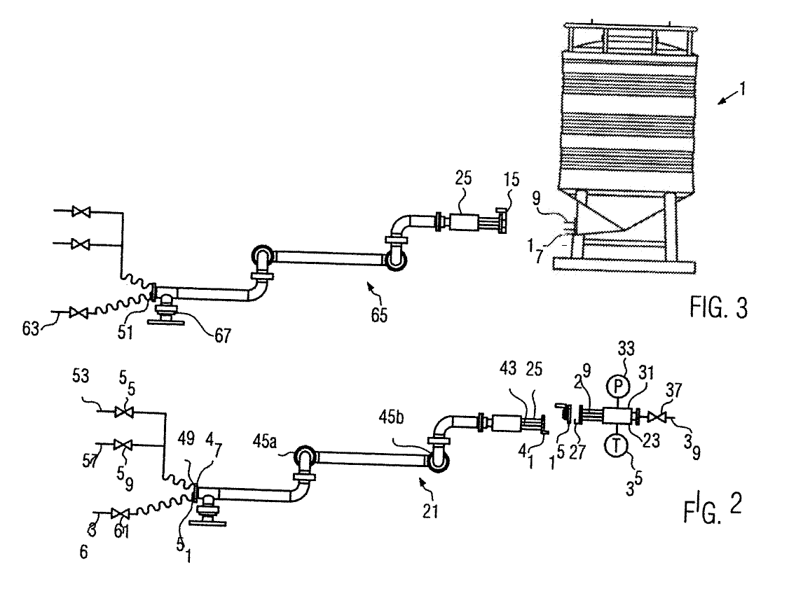

In accordance with an exemplary embodiment, compressed air feeding unit 57 and vapor feeding unit 53 can be used in first sterilization station 21. The compressed air from compressed air feeding unit 57 can be used to check the sealed arrangement of docking device 25, valve 15 and holding device 23. The vapor from vapor feeding unit 53 can be used for sterilization. Hydrogen peroxide feeding unit 63 is preferably used at a second sterilization station 65 shown in FIG. 3. Therefore, first connection point 49 can be used at first sterilization station 21 and second connection point 51 can be used at second sterilization station 65.

In the embodiment illustrated, vapor, for example, water vapor and/or hydrogen peroxide can be used for sterilizing. A benefit that hydrogen peroxide can provide is that germ-removal can occur at relatively low temperatures. Sterilization can thus be performed on heat-sensitive materials, such as, by way of non-limiting example, plastics. However, the sterilization period necessary for a low temperature sterilizing agent can be longer than a higher temperature sterilizing agent. Hydrogen peroxide can be provided in either liquid or gaseous form, whereby liquid hydrogen peroxide can evaporate upon contact with the walls that are previously heated at first sterilization station 21, and therefore preferably remove germs from the air as well.

Alterative sterilizing agent, such as, by way of non-limiting example, alcohol, acetic acid, can also be used, preferably in vapor form. Generally speaking, one of ordinary skill in the art would understand that any disinfectant that can be heated to over 100° C. that has been approved for food products can be used without deviating from the scope of the invention.

Referring to FIG. 3, second sterilization station 65 can sterilize the connection area, namely the interior of valve 15 and outlet connector 9 of liner 5. This sterilization process can be preferred because impure air can enter outlet connector 9 during docking. FIG. 3 shows the state in which valve 15 and outlet connector 9 have not been connected. As shown, both first sterilization station 21 and second sterilization station 65 have a common docking device 25, thus simplifying the system configuration.

Docking device 25 can be moved from first sterilization station 21 to second sterilization station 65, preferably with valve 15 attached to connecting assembly 41. Preferably, connecting assembly 41 and valve 15 form a seal, and valve 15 is moved toward outlet connector 9. Therefore, valve 15 can be brought to outlet connector 9 after being sterilized without the need for valve 15 to be removed from docking device 25. Therefore, the sterilization system provides valve 15 to be moved without the user touching valve 15, which can reduce the probability of valve 15 being contaminated subsequent to the first sterilization process. Referring to FIGS. 2-3, docking device 25 preferably includes a hinged joint 67 at which docking device 25 can be pivoted or otherwise displaced between first sterilization station 21 and second sterilization station 65. It is to be understood that alternate means for displacing docking device 25 between the sterilization stations 21, 65 can be provided as a matter of application specific design choice.

In accordance with an alternate embodiment, each sterilization station 21, 65 can include its own docking device rather than a shared docking device 25. In such an embodiment, it may be preferable for each sterilization station 21, 65 to have a single connection point through which the vapor, pressured air or sterilizing agent can be introduced.

At second sterilization station 65, hydrogen peroxide (H₂O₂) or other suitable sterilizing agent can be fed via the hydrogen peroxide feeding unit 63 via second connection point 51. Preferably bursting element 17 can withstand the sterilization process and does not burst at either first or second sterilization stations 21, 65. Examples of suitable material for bursting element 17 include plastic foils such as PE (polyethylene), HDPE (high density polyethylene), PP (polypropylene), HDPP (high density polypropylene) or PET (polyethylene terephthalate). Therefore, sterilization preferably is performed in a temperature range in which the plastic is not damaged, for example, softened.

In accordance with an exemplary embodiment, liquid hydrogen peroxide is sprayed into outlet connector 9 at which the hydrogen peroxide vaporizes on the walls that are still hot from the first sterilization process. It is also possible to use gaseous hydrogen peroxide without deviating from the scope of the invention.

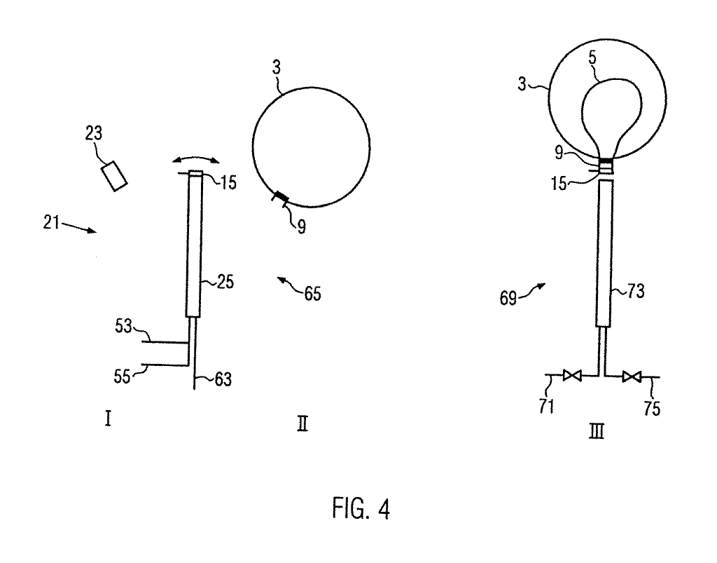

Reference is made to FIG. 4, wherein an embodiment of the sterilization system includes three sterilization stations 21, 65 and 69, indicated at stages I, II, and III respectively. Referring to stages I and II, docking device 25 has a valve 15 attached at an end thereof, and vapor feeding unit 53, compressed air feeding unit 55 and hydrogen peroxide feeding unit 63 at the opposite end of docking device 25. As indicated by the double headed arrow, docking device 25 can pivot such that the end of docking device 25 having valve 15 attached thereto can move between holding device 23 and outlet connector 9 of tank 3, thus moving between first sterilizing station 21 and second sterilization station 65.

A third sterilization station 69 can be provided at a separate location, for example, at a filling station for filling liner 5 with the product. At third sterilization station 69, the area from a product feeding unit 71 to valve 15, preferably in a closed position, can be sterilized. Depending on the user's preference and circumstances, for example, the duration of storage time of the bag-in-tank system 1, it may be preferable to clean and/or sterilize the connection area prior to filling liner 5 with the product. Additionally, the path to valve 15 can have contaminated air, or the conduit carrying the product or the exterior of valve 15 can be contaminated, thus rendering third sterilization station 65 desirable.

A second docking device 73 can be provided between valve 15 and a sterilizing agent feeing unit 75 and a product feeding unit 71. The sterilizing agent feeding unit can provide, by way of non-limiting example, water vapor, hydrogen peroxide or other suitable sterilizing agent. After the connection area is sterilized at third sterilization station 69, the product can be fed into liner 5 via product feeding unit 71 and through the sterilized second docking device 73. Preferably, as the product is being provided, the pressure of the product flow bursts bursting element 17 of liner 5, and the product can thereafter enter liner 5.

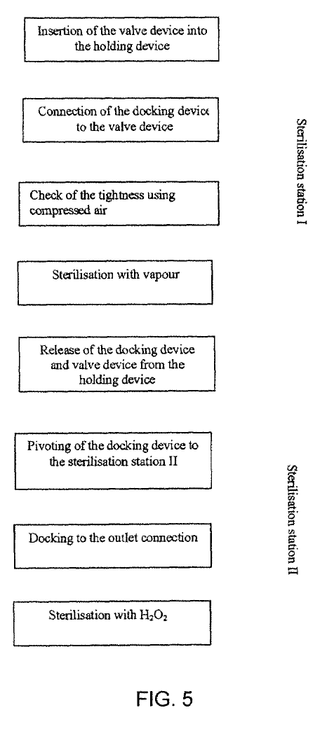

Reference is made to FIGS. 4-5, wherein an example of the process of sterilization in accordance with an embodiment of the system is described. Preferably, steps S1-S5 occur at first sterilization station 21 as illustrated in FIG. 2. In step S1, valve 15 in an open position is inserted into holding device 23. In step S2, docking device 25 is moved toward valve 15 and connected to valve 15 such that docking conduit 43, the opening of valve 15 and holder conduit 29 are aligned. The connection is preferably made such that docking device 25, valve 15 and holding device 23 form a sealed unit. By way of non-limiting example, one or more grippers can be provided to apply a force on docking device 25, valve 15 and holding device 23 toward each other after being assembled to secure the sealed unit. A plurality of washers can also be provided to enhance the tightness of the assembly.

The tightness of the assembly can then be tested at step S3, for example, using compressed air. Second valve 59 can be opened to permit the passage of compressed air into docking device 25, valve 15 and holding device 23. Pressure measuring device 33 of holding device 23 preferably measures the pressure to determine whether or not any air is lost, thus determining whether or not the tightness of the assembly is sufficient. Preferably, the compressed air is cleaned in compressed air feeding unit prior to being released into docking device 25.

Once the adequacy of the tightness of the assembly has been determined, sterilization with vapor, for example, water vapor, can be carried out in accordance with step S4. Preferably second valve 59 is closed to prevent further release of compressed air into docking device 25 and first valve 55 is opened. Hot vapor can be supplied into docking device 25 through first valve 55 to apply the hot vapor on the interior walls of docking device 25, valve 15 and holding device 23. Preferably, the temperature of the vapor is at least about 110° C., more preferably about 120° C. It is to be understood that any temperature that renders the vapor suitable for sterilizing valve 15 can be used without deviating from the scope of the invention.

Preferably, the temperature of the condensate forming at holding device 23 is measured using temperature sensor 35 at holding device 23, and the sufficiency of sterilization can be confirmed. For example, the duration of sterilization at a desired temperature can be measured. By way of non-limiting example, once the temperature of the condensate exceeds a certain threshold valve, the sterilization time begins. If the temperature of the condensate falls below the threshold, the sterilization time ends. If the temperature exceeds the threshold again, the sterilization time resets and begins again. Once the specified sterilization duration has elapsed at or above the threshold temperature, it can be an indication that sterilization has been completed. First valve 55 can be closed and air, for example, dry air, can be applied to docking device 25, valve 15 and holding device 23. Preferably, discharge valve 37 is open during sterilization and permits the condensate to drain into discharge 39.

In step S5, docking device 25 remains connected to valve 15 and is removed from holding device 23. Preferably, docking device 25 includes a holder (not shown) for retaining valve 15. A protective cap can also be placed on valve 15.

In step S6, docking device 25 having valve 15 attached thereto can be displaced, for example, pivoted, to second sterilization station 65. In step S7, valve 15 can be connected to outlet connector 9, preferably creating a sealed connection that is leak-proof. If a protective cap had been placed on valve 15, the protective cap is preferably removed shortly before valve 15 is connected to outlet connector 9. Valve 15 can be attached to outlet connector 9 or tank 3, with or without the use of grippers as matter of application specific design choice.

In step S8, the connection area can be sterilized, preferably up to and including the exposed surfaces of bursting element 17, using liquid or gaseous hydrogen peroxide, which can be provided from hydrogen peroxide feeding unit 63 via second connection point 51 by opening third valve 61. Preferably, liquid hydrogen peroxide can evaporate due to the high temperature of the walls. After a predetermined time has elapsed, the hydrogen peroxide sterilization process can be terminated. Subsequently, valve 15 can be closed and docking device 25 can be separated from valve 15. A protective cover of cap can be placed on valve 15 to protect valve 15.

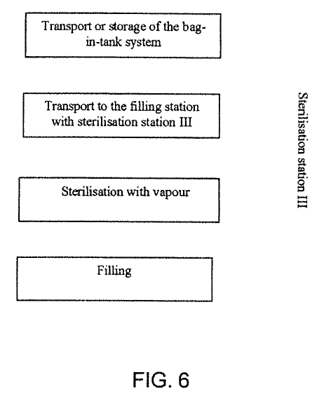

FIG. 6 demonstrates further steps taken in accordance with an embodiment having third sterilization station 69. Steps S9-S12 can be taken subsequent to steps S1-S8 of FIG. 5. Subsequent to sterilizing the connection area at second sterilization station 65, the bag-in-tank system 1 having valve 15 connected thereto, hereinafter referred to as the tank-valve assembly, can be moved to a subsequent station or stored. The tank-valve assembly can be transported to a filling station, in accordance with step S10, which preferably includes third sterilization station 69.

In step S11, second docking station 73 is sterilized up to the closed valve 15, thus sterilizing the outer surface of the closure of valve 15. Preferably, a vapor, more preferably water vapor at a temperature of greater than about 110° C., more preferably at about 120° C., is supplied into second docking station 73 by vapor feeding unit 75 via a corresponding valve. It is to be understood that vapor is not necessary, and hydrogen peroxide or other sterilizing agents can be used without deviating from the scope of the invention. Subsequently or coincidently, the connection area can be sterilized again by opening valve 15 and conducting the sterilization with valve 15 opened as a matter of application specific design choice.

In step S12, liner 5 is filled with the product being transported. Preferably, the corresponding valve of product feeding unit 71 is opened to permit the product to flow through second docking device 73 and into liner 5. The pressure of the product flow against bursting element 17 preferably causes bursting element 17 to burst open to permit the product to flow into liner 5.

Whereas the system has been described herein as a sterilization system, it is to be understood that the system can be used for disinfection or any other level of cleaning a system for filling a bag-in-tank system. For example, the sterilization stations 21, 65, 69 can be used for disinfection, rather than sterilization, or a combination of the two. One of ordinary skill in the art would understand that sterilization often refers to the destruction of germs and spores, to prevent the germs or spores from growing or multiplying in the culture medium represented by the product being transported, such as foodstuff.

By providing distinct sterilization stations 21, 65, 69, the system in accordance with an embodiment of the invention can facilitate removal of germs in areas that may often be difficult to access. For example, valves are often difficult to clean, especially the areas at which the valve lays against the outlet connector of the liner. In accordance with an exemplary embodiment, valve 15 is sterilized alone at first sterilization station 21, again at second sterilization station 65 when valve 15 is mounted on outlet connector 9, and a third time at third sterilization station 69 shortly before the liner is filled with the product. Additionally, all the areas that can contact the product are preferably sterilized.

The examples provided are merely exemplary, as a matter of application specific to design choice, and should not be construed to limit the scope of the invention in any way.

Thus, while there have been shown and described and pointed out novel features of the present invention as applied to preferred embodiments thereof, it will be understood that various omissions and substitutions and changes in the form and details of the disclosed invention may be made by those skilled in the art without departing from the spirit of the invention. It is the intention, therefore, to be limited only as indicated by the scope of the claims appended hereto.

It is also to be understood that the following claims are intended to cover all of the generic and specific features of the invention herein described and all statements of the scope of the invention which, as a matter of language, might be said to fall therebetween.

1-19. (canceled)

20. A sterilization system for sterilizing a connection area of a bag-in-tank system having a liner, an outlet connector and a valve, the sterilization system comprising:
a first sterilization station having a holding member constructed and arranged to retain a valve, the holding member having an anlysis chamber having a plurality of testing mechanisms;
the first sterilization station also having a first docking device constructed and arranged to contact the valve, the first docking device having first connecting point, wherein the first connecting point is connected to one or more first feeding units;
wherein the first docking device and the holding member contact the valve on opposite sides of the valve; and
a second sterilization station having a second docking device constructed and arranged to retain the valve and to displace the valve toward an outlet connector, the second docking device having a second conduit connected to a second connecting point, wherein the second connecting point is connected to one or more second feeding units.

21. The system of claim 20, wherein the first docking device and the second docking device is a common docking device.

22. The system of claim 21, wherein the common docking device is displaceable between the first sterilizing station and the second sterilizing station.

23. The system of claim 20, wherein the first feeding units comprise a vapor feeding unit and a compressed air feeding unit.

24. The system of claim 20, wherein the second feeding units comprise a hydrogen peroxide feeding unit.

25. The system of claim 20, wherein the testing mechanisms comprise a temperature sensor, a pressure measuring device and a discharging component.

26. The system of claim 20, wherein the first docking device comprises one or more hinged joints.

27. The system of claim 20, further comprising a third sterilization station having a third docking device constructed and arranged to selective receive a product flow from a product feed connecting point.

28. The system of claim 27, wherein the third docking device is connected to a sterilizing agent feeding unit.

29. The system of claim 20, wherein the first docking device comprises one or more hinged joints.

30. The system of claim 20, wherein the valve has an opening, the first docking device comprises a docking conduit and the holding device has a holding conduit; wherein the docking conduit, the valve opening and the holding conduit are aligned when assembled.

31. The system of claim 20, wherein the first docking device comprises one or more valves positioned between the first connecting point and the first feeding units.

32. The system of claim 20, wherein the second docking device is constructed and arranged to mount on the outlet connector while second docking device is connected to the valve.

33. A sterilization system for sterilizing a connection area of a bag-in-tank system for the transport of food products, wherein the bag-in-tank system includes a tank, a selectively removable liner constructed and arranged to be received in the tank, the liner having an outlet connector, and a selectively removable valve constructed and arranged to connect to the outlet connector, the sterilization system comprising:
a first sterilization station for sterilizing the valve, wherein the valve is disconnected from the tank; and
a second sterilization station for sterilizing the valve and the outlet connector in an assembled state.

34. The system of claim 33, wherein the first sterilization station comprises a holding device constructed and arranged to hold the valve, and a docking device constructed and arranged to provide a sterilizing agent.

35. The system of claim 34, wherein the docking device and the valve and the holding device form a seal when connected with the valve in an open position.

36. The system of claim 34, wherein the holding device comprises a temperature measuring device.

37. The system of claim 35, wherein the docking device comprises a vapor feeding unit for providing a sterilizing agent, the docking device further comprises a compressed air feeding unit for verifying the seal.

38. The system of claim 33, wherein the first sterilization station and the second sterilization station comprises a docking device.

39. The system of claim 33, further comprising a docking device constructed and arranged to be mounted on the outlet connector when the valve is attached to the docking device.

40. The system of claim 38, wherein the docking device comprises a hydrogen peroxide feeding unit.

41. The system of claim 33, further comprising a filling station for filling the liner, the system further comprising a third sterilization station for sterilizing product carrying areas of the filling station of the connection area.

42. A method of sterilizing a connection area of a bag-in-tank system having a tank, a selectively removable liner having an outlet connector, and a removable valve selectively connectable to the outlet connector, the method comprising:
providing a valve separated from the tank;
sterilizing the valve in a first sterilization station;
mounting the valve on the outlet connection; and
in a second sterilization station, sterilizing a connection area defined by the interior of the valve and the outlet connection in an assembled state.

43. The method of claim 42, further comprising
arranging the valve in a holding device;
connecting the valve to a docking device;
creating a sealed unit with the holding device, the valve and the docking device; and
sterilizing the connection area via the docking device.

44. The method of claim 43, wherein the sealed unit has a tightness, the method further comprising confirming the tightness by introducing compressed air into the docking device.

45. The method of claim 43, further comprising sterilizing the valve and sterilizing the connection area via the docking device.

46. The method of claim 43, further comprising mounting the valve and docking device in an assembled arrangement to the outlet connector.

47. The method of claim 42, further comprising sterilizing the valve includes applying a vapor having a temperature of at least about 110° C.

48. The method of claim 42, further comprising forming a condensate in the holding device, the condensate having a condensate temperature; and measuring the condensate temperature.

49. The method of claim 43, wherein sterilizing the connection area includes introducing hydrogen peroxide into the docking device.

50. The method of claim 42, further comprising transporting the bag-in-tank system to a filling station having a product-carrying area;
in a third sterilization station, sterilizing the product-carrying area and the connection area; and
filling the liner at the filling station.

51. The method of claim 50, wherein sterilizing the connection area includes applying vapor having a temperature of at least about 100° C.

52. A method of sterilizing a connection area defined by the interior of a connection assembly, the connection assembly including a valve and an outlet connector, the method comprising:
providing a valve having a first side and a second side;
creating a sealed unit by attaching the first side of the valve to a holding device having an analysis chamber and attaching the second side of the valve to a docking device connected to a feeding unit;
introducing compressed air into the docking device and measuring the pressure at the analysis chamber;
introducing a first sterilizing agent into the docking device and measuring the temperature at the analysis chamber;
detaching the first side of the valve from the holding device;
transporting the docking device attached to the valve toward the tank;
mounting the valve to the outlet connector; and
introducing a second sterilizing agent into the docking device.
